(19) United States
(12) Patent Application Publication
(10) Pub. No.: US 2007/0116775 A1
(43) Pub. Date: May 24, 2007

(54) PLASTIC CAVIAR BEAD FOR FILLING OF PILLOW HAVING FUNCTION OF ANTI-BACTERIA

(76) Inventor: Ho-Young Lee, Gyeonggi-Do (KR)

Correspondence Address: LEE, HONG, DEGERMAN, KANG & SCHMADEKA, 801 S. FIGUEROA STREET, 12TH FLOOR, LOS ANGELES, CA, 90017, US

(21) Appl. No.: 11/560,803

(22) Filed: Nov. 16, 2006

(30) Foreign Application Priority Data
Nov. 17, 2005 (KR) 10-2005-0110065

(51) Int. Cl. A61K 9/14

(52) U.S. Cl. 424/489

(57) ABSTRACT

The present invention relates to the plastic caviar bead for filling of pillow having function of anti-bacteria that combines the raw materials of synthetic resins of 100 in weight, antimicrobial of 0.5˜2 in weight, ceramic with hydrogen silicon and hydrogen aluminum of 3˜15 in weight that are pressed and corrected to the shape of granulation with 1‥5 mm in its diameter and having an excellent antibacterial function, it helps prevent all sorts of bacteria such as ticks to spread into the pillow while emitting atomic infrared rays that has a positive impact on human body and as well, its spherical shape increases the practicality of plastic caviar bead and enables a flow of air circulation by allowing the space between the beads and by emitting negative ion or atomic infrared rays, as well, the present invention of plastic caviar bead having a foam molding has an effect of light weight and superior comfort that the consumer want.

### CROSS-REFERENCE TO RELATED APPLICATIONS

This application is based on and claims priority to Korean Patent Application No. 10-2005-0110065 filed on Nov. 17, 2005 in the Korean Intellectual Property Office (KIPO), the entire contents of which are hereby incorporated by reference.

### FIELD OF THE INVENTION

The present invention relates to the plastic caviar bead for filling of pillow having function of anti-bacteria that combines the raw materials of synthetic resins of 100 in weight, antimicrobial of 0.5˜2 in weight, ceramic with hydrogen silicon and hydrogen aluminum of 3˜15 in weight are pressed and molded to the shape of granulation with 1˜5 mm in its diameter and with its excellent anti-bacterial function it helps prevent all sorts of bacteria such as ticks to spread into the pillow while emitting atomic infrared rays that has a positive impact on human body.

### DESCRIPTION OF THE RELATED ART

A pillow is a cushion which you rest your head on when you are in bed and it is generally comprised of a pillow cover with filings inside. The pillow cover is usually made of fabrics such as cotton and inside of the cover is stuffed with filling that keeps certain volume. As for filling, cotton made of synthetic fiber such as cotton, polyester, acrylic is used as well as pieces of fabric, feathers, sponge, red beans, mug beans, millet, hulled millet, grain, rice hulls and buckwheat chaff, etc.

However, these conventional types of filing are difficult to wash regularly that it produces a foul smell from human perspiration and especially, with cotton made of synthetic fibers, fabric pieces, feathers, etc., the weight is too light to support the neck and head of human body which results aches in the waist, neck, shoulder. As well, for the pillows made of grain or buckwheat chaff, if used for a long period, its friction creates not only the fine dust but also it spreads ticks, molds, bacteria if it is not washed and dried regularly which is quite inconvenient.

Recently, the synthetic resins chip has been invented for filling to solve this problem, however, although it is easy to wash and use, it is again quite heavy, hard and uncomfortable to use mainly due to its cylindrical shape,

On the other hand, silver is a type of copper that belongs to the periodic table No. 1B which has a brilliant shine of blue and white that has been used as a precious metal together with gold. After its unique function of sterilization has known, however, silver has been applied to many other areas, for example, by putting materials in the liquid of container made of silver, it helps to prevent the decay of things or by dropping a few drops of solution made of nitric acid to sterilize, it helps to prevent conjunctivitis of infants since long time. Thus, in recent years, there have been active researches on the various functions and effects of silver and especially, its unique nature of sterilization.

In particular, after the advent of Nano-technology, it is found that with very little amount of Nano silver (Ag) it is possible to sterilize materials very effectively and thus, it shows positive prospects in sterilization and prevention of bacteria by the use of Nano silver.

As such, after Nano Ag's superior effect of anti-fungal and sterilizing power was found, it has been used in all kind of daily products, however, there has been no attempt made using this for the plastic caviar bead for filling of pillow having function of anti-bacteria.

‘There is only negative ion that keeps our health in this industrial society today where the air pollution is becoming an alarming concern’ which the statement indicates a high interest in negative ion in relation to the health matters these days.

Negative ion is a scientific terminology that refers to a chemical component that is made of by adding more than one electron to atoms or molecules, in principal, all atoms and molecules can be altered to negative ion by acquiring electrons. That is, negative ion is corpuscle that has a negative electric charge that circulates in the air freely, thus it is very active and this active nature stimulates the metabolism of human cells and increases the energy and clears the blood system that helps recover from tiredness and improve the appetite. In other words, negative ion, in general, prevents the body becoming acidified and adjusts the blood vessel that is moved by autonomic nerves and internal organs to the favor of human body and stimulates the blood circulation by increasing the ion rate that is mineral components in the blood such as sodium, kalium that increase the strength of immune system to fight against virus in the blood as well as stimulates the metabolism of cells and, by producing endorphin and enkephalin, it also helps to recover from tiredness and improves the body strength. As mentioned above, there has been an increasing concern over health matters lately and thus, active researches and developments have been carried out in relation to the health products using negative ion that has a positive effect on human body. Examples of products made of negative ion are ranging from the accessories such as bracelet, necklace, belt using the materials such as charcoal, ocher, jade, titanium, tourmaline to the building materials, kitchen appliances and functional fabrics that combine negative ion material to resins and appointment to the fabric and also introduces other materials such as clothes, pillow, bedding, mate, etc made by using the above functional fabrics such as clothes.

### SUMMARY OF THE INVENTION

The pillow is usually made of fabrics such as cotton and is stuffed with filling inside that keeps certain volume. As for filling, cotton made of synthetic fiber such as cotton, polyester, acrylic is used as well as pieces of fabric, feathers, sponge, red beans, mug beans, millet, hulled millet, grain, rice hulls and buckwheat chaff. However, these conventional types of filing is difficult to wash regularly so that it produces a foul smell from the human perspiration and especially, with cotton made of synthetic fibers, fabric pieces, feathers, etc., the weight is too light to keep the neck and head of human body whereas pillows made of grain or buckwheat chaff are too heavy that it becomes impractical to use.

In order to overcome such shortcomings, the cylindrical shaped chip (1) using synthetic resins has been invented in recent years, which can be seen in the drawing 1, and it is used as filling inside of pillow cover (2), however, even though it is convenient to wash and use, due to its cylindrical shape, it feels hard and uncomfortable and also, because of its heavy weight, it is too heavy to use for adults that its usage has been confined to the children only.

In addition, for the products invented in relation to the ‘healthy pillow’ using negative ion, it is made by functional fabrics for the exterior of pillow that is made by the combination of negative ion material to resins and appointment to the fabric and for filling of pillow, it uses cotton made of synthetic resins such as polyester, acrylic, and fabric pieces, sponge, down, millet or buckwheat chaff, etc.

However, if the pillow is made as above, with negative ion material combined to the resins and appointment to the fabrics, there is a disadvantage of not being able to achieve a sufficient negative ion emitting effect.

### BRIEF DESCRIPTION OF THE DRAWINGS

The accompanying drawings, which are included to provide a further understanding of the invention and together with the description to explain the principle of the invention. In the drawings:

FIG. 1 is the pillow that contains the chip for the usage of filling in the pillow;

FIG. 2 is the perspective view and magnified sectional drawings that shows the plastic caviar bead;

FIG. 3 is the deal drawing showing the part of pillow that contains the plastic bead of the present invention for the plastic caviar bead for filling of pillow having function of anti-bacteria.

FIGS. 4-9 illustrate execution and test examples of the present invention.

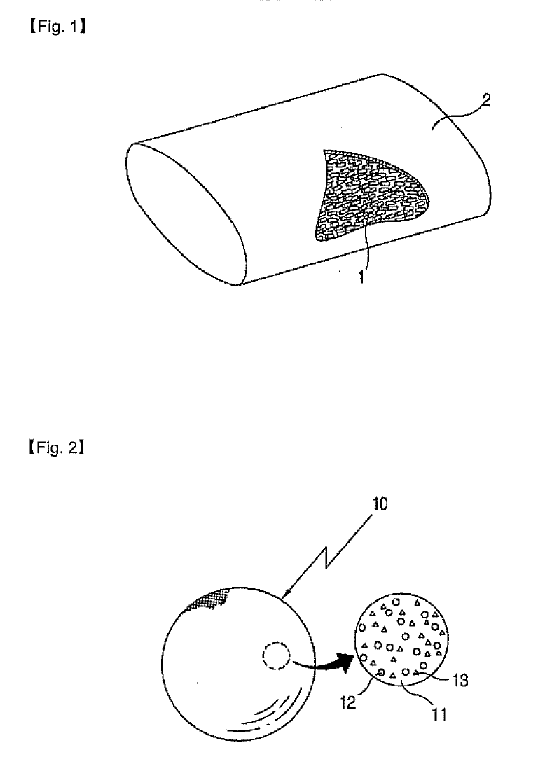

### DETAILED DESCRIPTION OF THE PREFERRED EMBODIMENTS

The present invention relates to the plastic caviar bead for filling of pillow having function of anti-bacteria that combines the raw materials of synthetic resins of 100 in weight, antimicrobial of 0.5˜2 in weight, ceramic with hydrogen silicon and hydrogen aluminum of 3˜15 in weight that are pressed and molded to the shape of granulation with 1˜5 mm in its diameter.

In addition, instead of above ceramic with main components of hydrogen silicon and hydrogen aluminum, it uses 3˜50 in weight of combined materials of one or more than two of ceramic elvan, jade and sericite with main components of hydrogen silicon and hydrogen aluminum that have characteristics of raw materials of atomic infrared rays.

Above synthetic resins is the olefin type resins such as polyethylene (PE), polypropylene (PP), and tourmaline powder of 1˜20 in weight is added then, pressed and molded.

Also, instead of tourmaline powder which is mentioned above, it combines the tourmaline powder and the emission ceramic powder in equal amount.

Also, it is formed and molded by the method of the above mentioned plastic caviar bead.

Also, anti-bacteria above has the characteristics of being inorganic anti-bacteria by using zerolite which is a synthetic product of non-surface area of more than 550 m² as a carrier which contains Nano Ag.

Also, instead of anti-bacteria above, it has the characteristics of using charcoal powder of 3˜20 in weight which is pressed and molded.

Hereafter, the detailed information on the manufacture, composition and effect on the present invention of the plastic caviar bead having the function of anti-bacteria is provided in accordance to the attached drawings.

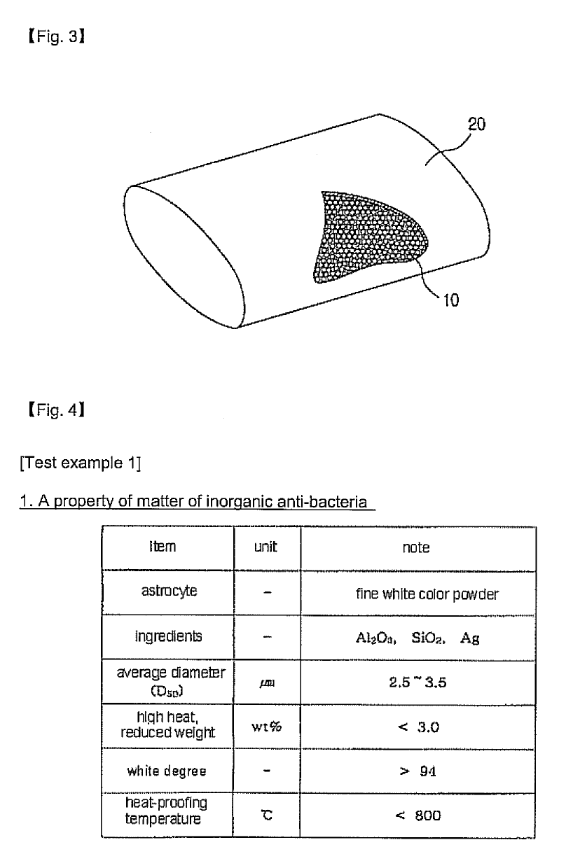

FIG. 2 is the perspective view showing the anti-bacterial plastic bead and magnified sectional Fig., and FIG. 3 is the deal drawing showing the parts of pillow that contain the plastic bead of the present invention.

The present invention relates to the plastic caviar bead for filling of pillow having function of anti-bacteria that combines the raw materials of synthetic resins of 100 in weight, antimicrobial of 0.5˜2 in weight, ceramic with hydrogen silicon and hydrogen aluminum of 3˜15 in weight that are pressed and molded to the shape of granulation with 1˜5 mm in its diameter.

The raw materials of synthetic resins of present invention can be all types of resins; especially the usage of polyethylene (PE), polypropylene (PP), and polystyrene (PS) type of resins are ideal to use.

The use of hydrogen silicon and hydrogen aluminum is most ideal for ceramic above, so the present invention uses synthetic atomic infrared rays' emitter having the main components of high degree of purified hydrogen silicon and hydrogen aluminum. However, for the manufacture of filling of pillow having the ant-bacteria that emits atomic infrared rays, it can also use atomic infrared rays' emitter such as clay, loess, kaolinite, raw ore pulverization, jade, elvan, etc.

The present invention relates to the plastic caviar bead for filling of pillow having function of anti-bacteria that combines the raw materials of synthetic resins of 100 in weight, antimicrobial of 0.5˜2 in weight, and ceramic with hydrogen silicon and hydrogen aluminum of 3˜15 in weight.

In case of combining the anti-bacteria below 0.5 in weight, it has little anti-bacterial effect and if combined more than 2 in weight, it becomes too expensive to be economical, thus the combined quantity has to be adjusted within the range of 0.5˜2 in weight. Anti-bacteria used in the present invention can use either inorganic or organic, or both at the same time. For organic anti-bacteria, the general organic type ant-bacteria is used by the method of spraying it, however, if organic type anti-bacteria is used for a long period it has a disadvantage of not being able to keep the effectiveness of anti-bacterial function, thus the effect of its use is more apparent when it is used within a short period of time.

in addition, if the mix ratio of ceramic is below 3 in weight, it reduces the atomic infrared rays emitting ability so that it can not be claimed that it has an atomic infrared rays emitting ability, however, if it is more than 15 in weight, it boosts the atomic infrared rays emitting ability but the strength between the raw materials of synthetic resins is reduced so that it is difficult to form filling of pillow to the shape of granulation. Therefore, the mix ratio of ceramic above should be adjusted within the range of 3˜15 in weight when it uses the raw material synthetic resins of 100 in weight.

The main components of synthetic resins that are combined by the ratio above, anti-bacteria and ceramic are combined and then, pressed and molded to the shape of granulation. In this case, there is no particular method of molding it but use the general molding equipment and method. There is also no restrictions on the shape of granulation, however, to mold to the spherical shape is easy to use for filling of pillow, and the size of granulation is preferred to be 1˜5 mm in its diameter as it is easy to process and also comfortable to use.

Also, instead of ceramic above having the main components of hydrogen silicon and hydrogen aluminum, it uses the materials of atomic infrared rays of 3˜50 in weight which is the combination of one or more than two of ceramic elvan, jade and sericite having main components of hydrogen silicon and hydrogen aluminum.

The atomic infrared rays material that is used in the present invention is the combination of one or more than two of ceramic, elvan, jade and sericite, and when the raw materials of synthetic resins of 100 in weight is used as a basis, it combines and mixes the synthetic resins atomic infrared rays material of 3˜50 in weight. In this case, the combined atomic infrared rays materials such as ceramic, elvan, jade and sericite is preferred in a powder form. Especially, ceramic having main components of hydrogen silicon and hydrogen aluminum are the best. The present invention uses synthetic atomic infrared resins emitter having main components of high degree of purified hydrogen silicon and hydrogen aluminum, loess, kaolinite, etc.

The combination ratio of raw materials of synthetic resins, anti-bacteria and atomic infrared rays that are used in the plastic caviar bead for filling of pillow having function of anti-bacteria has to be atomic infrared rays of 3˜50 in weight when the raw materials of synthetic resins of 0.5˜2, in weight is used as the basis.

In addition, if the mix ratio of ceramic is below 3 in weight, it reduces the atomic infrared rays emitting ability so that it can not be claimed that it has the atomic infrared rays emitting ability, however, if it is more than 50 in weight, it boosts the atomic. infrared emitting ability but the strength with the raw materials of synthetic resins is reduced that it is difficult to form filling of pillow having the shape of granulation. Therefore, the mix ratio of ceramic above should be adjusted within the range of 3˜15 in weight when it uses the raw material synthetic resins of 100 in weight.

Synthetic resins that are combined in accordance to the ratio above, anti-bacterial and ceramic are combined and then, pressed and molded to the shape of granulation. In this case, there is no particular method of molding it but use the general molding equipment and method.

Above synthetic resins is the olefin type resins such as polyethylene (PE), polypropylene (PP), and tourmaline powder of 1˜20 in weight is added then, pressed and molded.

The raw materials of resins that are used in the present invention are polyethylene (PE), polyvinylchloride (PVC), polystyrene (PS), polypropylene (PP) as well as other common thermoplastics, however, it is ideal to use polyethylene (PE) or polypropylene (PP) which uses olefin resins as the main source that has no harming effect on human body, especially, in case of using polyethylene (PE), since it is solid because of its hardness of the material, it is best to use the HDPE (high-density polyethylene) that has low humidity transmissivity.

Tourmaline that is used in the present invention has crystallization of six pillar-shaped with the chemical elements of iron, magnesium, alkali materials and etc. as well as the complicated aluminum of borosilicate which is a natural mineral belonging to the hexagonal system. It is 7.0˜7.5 in solidity and 2.98˜3.20 in weight, and it has glass luster or resinous luster. The electrics are charged by the friction of tourmaline and when heated, it is charged to the positive pole and negative pole in each end. Because it is charged with electricity, it has the name of tourmaline.

The crystallization of tourmaline produces a continual aphrodisiac electric current that works to moisture contained in the air so that it decomposes H₂0 and separates a hydrogen ion (H⁺) and an oxalic acid ion (OH⁻) which make negative ion.

Tourmaline having the characteristics of producing negative ion, having a surface active function and also emitting atomic infrared rays of 4˜14 micron has been applied to the environment and health products such as fabric, cosmetics, food, plastic, ceramic, housing, water treatment, livestock faring, agricultural industry among many others starting from the 1990s.

For the present invention, if the combined amount of tourmaline powder to olefin resins of 100 in weight is below 1 in weight, the produced plastic bead has little negative ion emitting effect, and if it exceeds 20 in weight, in comparison to the plastic beads that uses an expensive tourmaline as the material, the efficiency of negative ion emit is not great enough to compensate the expense occurred, thus it is not favorable in terms of economic value. Whereas, if more than 20 in weight of tourmaline are combined, because the adhesiveness is reduced, it is easy to break resins that are highly impractical.

Also, if the combination ratio of anti-bacteria is below 0.5 in weight in comparison to olefin resins above of 100 in weight, it has no effect on anti-bacterial capability of plastic bead and if the combination ratio is more than 2 in weight, in comparison to 2 in weight, the effect on anti-bacterial capability does not show much difference.

Therefore, the amount of tourmaline powder combined to manufacture the plastic caviar bead for filling of pillow having function of anti-bacteria, it is ideal to adjust within the range of 1˜20 in weight each and 0.5˜2 in weight to the ratio of olefin resins of 100 in weight

Also, instead of tourmaline powder above, it has the characteristics of mixing the combination of tourmaline powder and emitting ceramic powder in equal amount.

According to the present invention, instead of using tourmaline powder, it can also use the combination of tourmaline powder and emitting ceramic powder in equal amount. Emission ceramic powder used in the present invention means atomic infrared rays, negative ion emitter and ceramic powder with deodorization effect. For this, it is best to use ceramic having main components of hydrogen silicon and hydrogen aluminum and the present invention uses synthetic ceramic that has a high degree of purified hydrogen silicon and hydrogen aluminum.

Also, the above plastic bead has a characteristic of being formed and molded by the method mentioned above.

The above plastic bead of the present invention being formed and molded by the method mentioned above reduces the weight and provides the plastic bead having anti-bacterial negative ion that is light and comfortable which satisfies the consumers' need and also improves the practicality of plastic beads in accordance to the present invention.

That is, combine and mix olefin type resins of 100 in weight such as polyethylene (PE), polypropylene (PP), tourmaline powder or the mix of tourmaline powder and emitting ceramic powder in equal amount of 1˜20 in weight, and anti-bacteria of 0.5˜2 in weight, after then, based on the total weight of synthetic resins, add chemical forming agent of 1-5% in weight and press and mold using the method mentioned above to the shape of granulation plastic bead with 1-5 mm in its diameter.

Here, if it adds 1% in weight of above chemical formation and to be pressed and molded, the weight of plastic bead of the present invention is decreased by up to 0.7, and if it adds 5 weight %, the weight is reduced below 0.1. In other words, if 1% in weight of above chemical formation is added, the degree of reduction in weight is too little and if it exceeds 5%, it becomes too light to use, thus it is ideal to add 1˜5% in weight of above chemical formation.

Also, anti-bacteria above has the characteristics of being inorganic anti-bacteria by using zerolite which is a synthetic product of non-surface area of more than 550 m² as a carrier which contains Nano Ag.

Inorganic anti-bacteria that is used in the present invention uses zerolite as a carrier, it uses inorganic anti-bacteria which contains Nano Ag. That is, inorganic anti-bacteria above is dispersed by Nano Ag that has a strong sterilization effect on the surface of zeolite. This zeolite is good to use synthetic resins of non-surface area of more than 550 m² and it can also combine zinc as an antifungal metal or it can be used on its own.

Also, the present invention has a characteristic of using charcoal powder of 3˜20 in weight instead of anti-bacteria above and is pressed and molded.

Charcoal has a strong effect on anti-bacterial and negative ion that is used for the variety of health products. By adding charcoal powder to the present invention it can maximize the effect of anti-bacterial and negative ion emittance. Charcoal is best used as a powder form as it is easy to use.

Additional explanation may be offered in relation to the effect of negative ion of charcoal: having the function of blood purification, negative ion neutralize the acidity blood and change it to a weak alkali which has a resisting power and helps prevent geriatric diseases, cancer, allergy, etc. As well, negative ion stimulates parasympathetic nerve so it relaxes mind and body while activating beta endorphin that amplifies the feeling of happiness. In addition, by having an autonomic nerve settlement function, negative ion deduced from charcoal plays the role of balancing the symptoms that users suffer today such as insomnia, headache, feeling of cold, menopause disease, shoulder aches, lumbago, inveterate fatigue and etc. and by strengthening its immune system, it also strengthen the intestines and liver and thus helps enjoy a healthy life.

### Execution and Test Examples

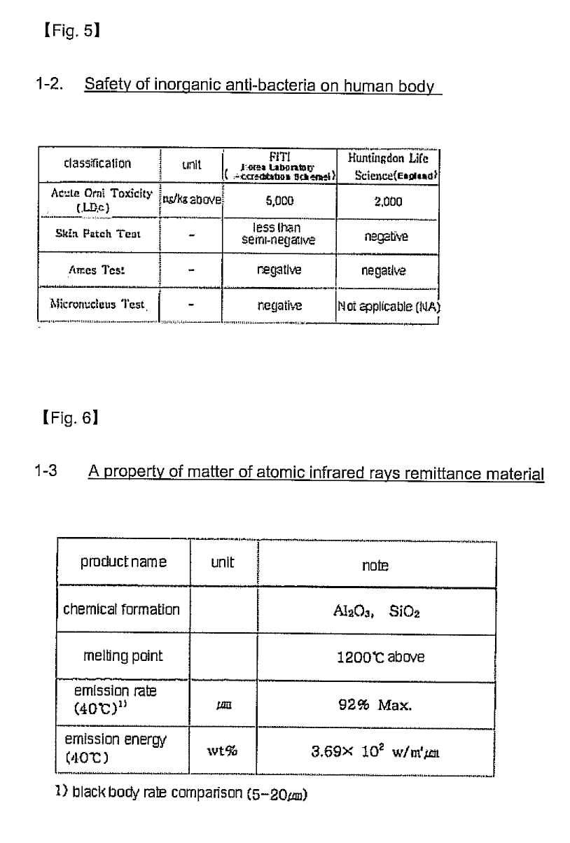

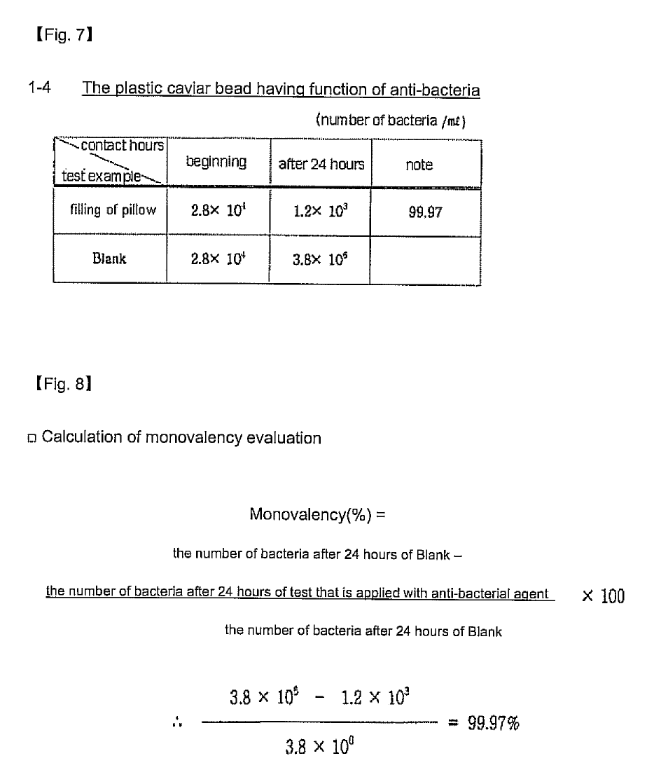

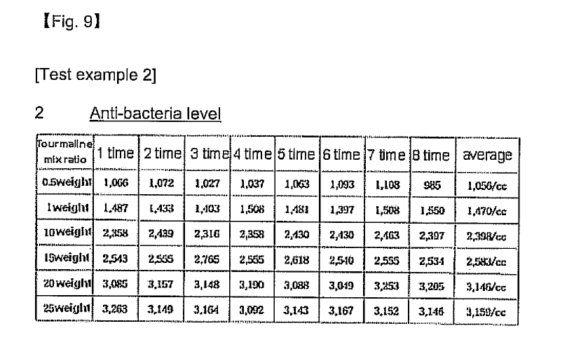

FIGS. 4-9 illustrate execution and test examples to provide a further understanding of the present invention and together with the description to highlight the effectiveness of the present invention. In reference to the tests included in the appendices, additional explanation are offered for the process and completion of pillow using various methods and materials for the plastic caviar bead having function of anti-bacteria.

In reference to the execution example 1, the manufacture of atomic infrared rays emittance plastic bead, the combination of the polyethylene (PE) of 100 in weight, inorganic anti-bacteria by using zerolite which is a synthetic product of non-surface area of more than 550 m² as a carrier which contains Nano Ag and synthetic resins ceramic of 15 in weight having main components of high degree of purified hydrogen silicon and hydrogen aluminum has been used. After mixing the raw materials above, it is formed and molded to the shape of granulation with 1.2 mm in its diameter.

In reference to the execution example 1-1, making the pillow and also illustrated in drawing 3, the manufacture of pillow is completed by inserting the atomic infrared rays emittance plastic bead (10) that is manufactured in accordance to the present invention described above, inside of pillow cover (20) that is made of cotton fabric.

In reference to the test example 1, a property matter of inorganic anti-bacteria, the measurement of characteristic for inorganic anti-bacteria that is used in the present invention by containing Nano Ag on the surface of zerolite is shown in appendix I;

In reference to the test example 1-2, the safety of inorganic anti-bacteria to human body, which the test result has been also checked by the Korea Consumption Science Research Center and Huntingdon Life Science in England upon request, is shown in appendix II.

In reference to the test example 1-3, a property matter of atomic infrared rays emittance material, the measurement of characteristic of synthetic resins ceramic that is synthetic atomic infrared resins emitter having main components of high degree of purified hydrogen silicon and hydrogen aluminum is shown in appendix III.

In reference to the test example 1-4, the plastic caviar bead having function of anti-bacteria, the plastic caviar bead having function of anti-bacteria, that is testing of bacteria after applying anti-bacterial and measure the anti-bacterial level (, the result is shown in appendix IV: (Test method: pressurized application, test colicin: a colon bacillus (Escherichia Coil ATCC 25922)).

The results in the execution and test examples above demonstrate that plastic caviar bead having function of anti-bacteria manufactured in accordance to the present invention has a superb anti-bacteria level.

The pillow made of plastic caviar bead having function of anti-bacteria of the present invention has a good air ventilation, an excellent anti-bacteria function, and by emitting atomic infrared rays that is suitable to human body, it works favorably to the human body of user and has characteristics of enabling sound sleep, getting rid of smell (deodorization) and heavy metal, stimulating metabolism, preventing fatigue and all other common diseases of adults, etc and thus it helps improving the health condition of users on the whole.

In reference to the execution example 2, the manufacture of the plastic caviar bead for filling of pillow having function of anti-bacteria, three different methods of combining and mixing the raw material for the plastic bead having the function of anti-bacteria have been carried out.

First, combine and mix the high density polyethylene of 100 in weight, tourmaline powder 15 in weight, inorganic anti-bacteria of 1 in weight which disperses Nano Ag on the surface of more than 550 m² zerolite and form and mold to the shape of granulation with 3 mm in its diameter.

Second, combine and mix the high density polyethylene of 100 in weight, mixture of tourmaline powder and emittance ceramic powder in equal amount of 15 in weight, inorganic anti-bacteria of 1 in weight which disperses Nano Ag on the surface of more than 550 m² zerolite and form and mold to the shape of granulation with 3 mmin its diameter.

Third, combine and mix the high density polyethylene of 100 in weight, tourmaline powder of 15 in weight, inorganic anti-bacteria of 1 in weight which disperses Nano Ag on the surface of more than 550 m² zerolite and add C709 (Dongjin Semicamsa Product) as a chemical emitter of 3 in weight, based on the weight of above resins mixture, and to be pressed and molded to the shape of granulation with 3 mmin its diameter.

In reference to the execution example 2-1, making of pillow, as illustrated in drawing 3, complete the making of pillow by inserting the atomic infrared rays' emittance plastic bead (10) that is manufactured in accordance to the above method, inside of pillow cover (20).

As in the test example 2-1 above, the manufacture of plastic caviar bead for filling of pillow having function of anti-bacteria uses negative ion emittence material that is made of 1) tourmaline, 2) the mixture of tourmaline powder and the emitting ceramic powder in equal amount, 3) tourmaline but add chemical emitter then to be press and molded, after then, test negative ion emittance amount five times each and deduce the average of 1) 2,663/cc, 2) 2,585/cc, 3) 2,543/cc each which show a little variance among different materials used.

On the other hand, at the molding stage 3) above that is formed and molded in accordance to the method above, has a weight of about 0.2 which is only made by forming and molding without adding emittance and when it is compared to the plastic beads of 1) and 2), it is much lighter and superior in comfort.

The plastic caviar bead for filling of pillow having function of anti-bacteria in accordance to the present invention is light, elastic, comfortable and has a superb effect on atomic infrared rays or negative ion emittence.

In reference to the test example of 2, negative ion emittance quantity according to the mixture of tourmaline, it combines and mixes the high density polyethylene of 100 in weight, inorganic anti-bacteria of 1 in weight which disperses Nano Ag on the surface of more than 550 m² zerolite and tourmaline powder of 0.5 in weight, 1 in weight, 10 in weight, 15 in weight, 20 in weight, 25 in weight and then, to be formed and molded to the shape of granulation with 5 mm in its diameter and complete the plastic caviar bead for filling of pillow having function of anti-bacteria.

This test was conducted on the clear day 10˜11 am, 25□, humidity 40˜50, negative ion 25˜50/cc and use ion tester COM3010 PRO (Com System, Inc product).

By differing the quantity of tourmaline above, it measures 6 times each of the negative ion emmitance quantity of the plastic caviar bead for filling of pillow having anti-bacteria negative ion and the result showing the average is included in appendix V.

As can be seen in the table of appendix V, for the plastic caviar bead for filling of pillow having anti-bacteria negative ion, it is confirmed that when the combination rate of tourmaline is 0.5 in weight, 1 in weight, 10 in weight, 15 in weight, 20 in weight, 25 in weight, it emits negative ion of 1,056/cc, 1,470/cc, 2,398/cc, 2,583/cc, 3,146/cc, 3,159/cc on average each and thus the higher quantity of tourmaline is combined, the higher rate of negative ion emittance will occur.

However, negative ion emittance, when tourmaline is combined 0.5 in weight, is 8 times average 1,056/cc, which in comparison to the combination of 1 in weight of 8 times average 1,470/cc, negative ion emittance is so much lesser that it has big differences in negative ion emittance quantity, and when tourmaline is combined 25 in weight, negative ion emittance amount is 8 times average 3,159/cc which, in comparison to the combination of 20 in weight results 8 times average of 3,146/cc which shows little variance in negative ion emittance quantity.

Therefore, the ideal mix ratio of tourmaline to manufacture the plastic bead for filling of pillow having anti-bacteria negative ion emittance is confirmed to be 1˜20 in weight.

On the other hand, to HDPE 100 in weight, combine tourmaline 4 in weight and anti-bacteria 1 in weight, then measure negative ion emittance quantity of average 8 times which is 1,747/cc and reduction of bacteria is 99.7%.

Effects of the Present Invention

The plastic caviar bead for filling of pillow having function of anti-bacteria in accordance to the present invention is made of synthetic resins that has a proper elasticity thus, when used as filling of pillow, it supports the neck and the head of user comfortably that helps recover from the tiredness, and also because of its granulation shape, it enables an easy flow of air ventilation, it feels cool and also easy to wash. In addition, the present invention uses the plastic caviar bead for filling of pillow having function of anti-bacteria that plays not only the role of comfortable bedding but because of its superb anti-bacterial function, it can also prevent the spread of bacteria such as ticks while providing a plenty of atomic infrared rays and negative ion that are beneficial to human body thus, it has an effect of improving the health of user.

Also, the plastic caviar bead for filling of pillow having function of anti-bacteria bacteria in accordance to the present invention is a natural mineral which has an effect on negative ion emittance that is the characteristics of tourmaline so while sleeping, the volume of negative ion is increased which in turn, increases the activity of cells that boost the metabolism between the cell walls of materials which helps the movement of cells so that it improves the blood circulation and in turn, leads to a sound sleep, accordingly, it has an effect of recovery from the tiredness and improves the general health, and by adding the anti-bacterial agent, it also has an incidental effect on getting rid of all sorts of bacteria without sterilizing it.

Also, the plastic caviar bead for filling of pillow having function of anti-bacteria in accordance to the present invention is molded to the shape of spherical, it can further increase the practicality of plastic bead and by using it as filling of pillow that helps air ventilation between the space of bead, it feels cool. As such negative ion or atomic infrared rays have a positive effect on human body on the whole and the plastic caviar bead that is pressed and molded can provide the light weight and a superior comfort to the consumers.

What is claimed is:

1. An anti-bacteria plastic caviar bead for filling a pillow, the bead comprising:
synthetic resins of 100 in weight;
one of antimicrobial of 0.5˜2 in weight and charcoal powder of 3˜20 in weight; and
ceramic with hydrogen silicon and hydrogen aluminum of 3˜15 in weight,
wherein the synthetic resins, one of antimicrobial and charcoal powder and ceramic are pressed and molded into a granulated shape of 1˜10 mm in diameter.

2. The anti-bacteria plastic caviar bead of claim 1, wherein the resins comprise olefin type resins of 1˜20 in weight that are pressed and molded.

3. The anti-bacteria plastic caviar bead of claim 2, wherein the olefin type resins comprise one of polyethylene (PE), polypropylene (PP) and tourmaline powder.

4. The anti-bacteria plastic caviar bead of claim 2, wherein the olefin type resins comprise a combination of tourmaline powder and emitting ceramic powder in equal amounts.

5. The anti-bacteria plastic caviar bead of claim 1, wherein the ceramic has the characteristics of raw materials of atomic infrared rays and further comprises at least one of ceramic elvan, jade and sericite.

6. The anti-bacteria plastic caviar bead of claim 5, wherein the resins comprise olefin type resins of 1˜20 in weight that are pressed and molded.

7. The anti-bacteria plastic caviar bead of claim 6, wherein the olefin type resins comprise one of polyethylene (PE), polypropylene (PP) and tourmaline powder.

8. The anti-bacteria plastic caviar bead of claim 6, wherein the olefin type resins comprise a combination of tourmaline powder and emitting ceramic powder in equal amounts.

9. The anti-bacteria plastic caviar bead of claim 1, wherein the antimicrobial comprises more than 550 m² synthetic zerolite that contains Nano Ag such that the antimicrobial has characteristics of inorganic antimicrobial that contains Nano Ag.

10. The anti-bacteria plastic caviar bead of claim 9, wherein the ceramic has the characteristics of raw materials of atomic infrared rays and further comprises one or more of ceramic elvan, jade and sericite.

11. A pillow having a filling of anti-bacteria bead, the anti-bacteria bead comprising:
synthetic resins of 100 in weight;
one of antimicrobial of 0.5˜2 in weight and charcoal powder of 3˜20 in weight; and
ceramic with hydrogen silicon and hydrogen aluminum of 3˜15 in weight, wherein the synthetic resins, one of antimicrobial and charcoal powder and ceramic are pressed and molded to a shape of granulation of 1˜10 mm in diameter.

12. The pillow of claim 11, wherein the resins comprise olefin type resins of 1˜20 in weight that are pressed and molded.

13. The pillow of claim 12, wherein the olefin type resins comprise one of polyethylene (PE), polypropylene (PP) and tourmaline powder.

14. The pillow of claim 12, wherein the olefin type resins comprise a combination of tourmaline powder and emitting ceramic powder in equal amounts.

15. The pillow of claim 11, wherein the ceramic has the characteristics of raw materials of atomic infrared rays and further comprises at least one of ceramic elvan, jade and sericite.

16. The pillow of claim 15, wherein the resins comprise olefin type resins of 1˜20 in weight that are pressed and molded.

17. The pillow of claim 16, wherein the olefin type resins comprise one of polyethylene (PE), polypropylene (PP), and tourmaline powder.

18. The pillow of claim 16, wherein the olefin type resins comprise a combination of tourmaline powder and emitting ceramic powder in equal amounts.

19. The pillow of claim 11, wherein the antimicrobial comprises more than 550 m² synthetic zerolite that contains Nano Ag such that the antimicrobial has characteristics of inorganic antimicrobial that contains Nano Ag.

20. The pillow of claim 19, wherein the ceramic has the characteristics of raw materials of atomic infrared rays and further comprises one or more of ceramic elvan, jade and sericite.
